(19) United States
(12) Patent Application Publication
(10) Pub. No.: US 2022/0145265 A1
(43) Pub. Date: May 12, 2022

(54) IN VITRO EQUINE MODEL SYSTEMS AND THEIR INTEGRATION INTO HORSE-ON-A-CHIP PLATFORM

(71) Applicant: University of Kentucky Research Foundation, Lexington, KY (US)

(72) Inventor: Carrie L. Shaffer, Lexington, KY (US)

(21) Appl. No.: 17/522,619

(22) Filed: Nov. 9, 2021

Related U.S. Application Data
(60) Provisional application No. 63/111,349, filed on Nov. 9, 2020.

(51) Int. Cl. C12N 5/071; C12M 3/06; C12M 1/00; C12M 1/12

(52) U.S. Cl. CPC C12N 5/0697; C12N 5/0679; C12N 5/0688; C12M 23/16; C12N 2501/10; C12M 25/02; C12N 2506/45; C12N 2506/02; C12M 23/26

(57) ABSTRACT

In vitro equine organ model systems, and methods of making and using such systems, are provided and can include an organoid prepared using equine tissue associated with the organ of interest; or equine primary cells, wherein the equine primary cells are derived from equine tissue associated with an organ of interest, or derived from an organoid prepared using equine tissue associated with the organ of interest.

### RELATED APPLICATIONS

This application claims priority from U.S. Provisional Application Ser. No. 63/111,349 filed Nov. 9, 2020, the entire disclosure of which is incorporated herein by this reference.

### TECHNICAL FIELD

The presently-disclosed subject matter generally relates to equine organoids, microfluidic chip systems, and related applications. In particular, certain embodiments of the presently-disclosed subject matter relate to equine organoids and in vitro equine organ model systems including a monolayer of differentiated cell types derived from three-dimensional organs prepared using equine tissue associated with one or more organs of interest. Such organs of interest include, for example, lung, trachea, stomach, intestine, liver, bile duct, kidney, bone, skin, pancreas, cecum, colon, brain, neuron, salivary gland, retina, placenta, uterus, and mammary gland.

### INTRODUCTION

The study of equine disease has been significantly limited by the lack of in vitro models that accurately reflect the dynamic physiology of the horse. Recent advances in human medicine have led to the development of microscopic, organ-like model systems (termed ‘organoids’ and ‘spheroids’) that recapitulate intricate organ architectures and functionalities. Remarkably, such three-dimensional (3D) in vitro organoids have enabled scientists to model difficult-to-culture cancers and address fundamental questions in cell and developmental biology, infectious disease mechanisms, and the impact of genetic abnormalities. In addition, organoids have emerged as an invaluable tool for accurately predicting drug metabolism and response, and represent an ideal platform for therapeutic discovery and pre-clinical development.

However, limited progress has been made towards the development of equine organoids. To date, the development of equine organoids has been limited to the generation of ‘mini-guts’ that have provided an advanced system in which to study intestinal cell dynamics and disease states.

Furthermore, the physiologic environment within an animal is dynamic. As such, additional levels of control would be needed to recreate the dynamic physiologic environment within a horse to provide robust in vitro equine model. Mechanical forces are critical in biology and serve to drive gene expression, cellular function, cell shape, and tissue architecture.

Thus, there is a pressing need to develop new in vitro systems that recapitulate diverse equine tissue and organ microenvironments. The lack of such systems limits the ability to evaluate conditions, diseases, and treatments; limits the ability to mimic animal tissue and diverse organ systems; makes it difficult to reflect native organ function; and prevents the ability to investigate multi-organ effects. Prevalence of equine industries, including equine racing, gives rise to a need for more reliable, comprehensive, and ethical animal testing methods.

### SUMMARY

The presently-disclosed subject matter meets some or all of the above-identified needs, as will become evident to those of ordinary skill in the art after a study of information provided in this document.

To address the unmet needs in the art, disclosed herein is a sustainable, biologically-relevant equine organoid systems that exhibit organ- and tissue-specific characteristics. For example, disclosed herein is an equine airway organoid system that exhibits lung- and trachea-specific characteristics, including coordinated ciliated cell beating and mucus production. For additional examples, also disclosed herein are in vitro equine hepatic/liver, renal/kidney, and gastrointestinal organoids. Also disclosed herein are unique methods for preparing replenishable in vitro equine organoid tissues that maintain features of native organs. Additional in vitro organoid and equine organ model systems are disclosed herein, and related methods with will apparent to the skilled artisan upon study of this document.

In order to incorporate important biomechanical forces that influence cellular architecture and recapitulate diverse tissue microenvironments, they engineered organoid systems disclosed herein are translated into an innovative ‘horse-on-a-chip’ microfluidic platform that affords additional precision and control of biomechanical forces that recapitulate organ function and recreates the dynamic physiologic state in which tissues function in vivo.

For example, disclosed herein is an equine lung-on-a-chip, liver-on-a-chip, kidney-on-a-chip, and small intestine-on-a-chip, and other organ-on-a-chip systems. Such systems can facilitate a variety of investigations that are currently not feasible in the horse.

As disclosed herein, generation of diverse equine organoids and translation of organoid tissue into physiologically-relevant microfluidic devices will enable transformative research in multiple areas of equine health and disease. A robust, biomimetic equine platform as disclosed herein can facilitate high-impact studies with immediate translational potential, for example, by addressing outstanding questions in equine infectious disease, therapeutic and antimicrobial development, allergic and immune-modulated conditions, tissue remodeling in response to injury, and developmental biology.

Ultimately, the use of equine organoids and biomimetic microfluidic organ-chip systems will reduce experimental animal testing and will open new avenues to explore mechanisms underlying infectious disease dynamics, tissue plasticity and morphogenesis, metabolism and therapeutic discovery, inflammation, and host-microbiome interactions.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1F. Equine respiratory basal cells self-assemble into 3D spheroid structures. FIG. 1A: Proximal respiratory organoids self-assemble and form a hollow lumen in 3D culture at the air-liquid interface. FIG. 1B: An example of a ‘apical-out’ organoid that has spontaneously differentiated in reversed polarity. FIG. 1C: Differential interference microscopy (DIC) of a mature branched organoid reveals an air sac-like morphology. FIG. 1D: Mucus-producing tracheospheres develop within five days of culture. In FIGS. 1A-1D, the scale bar represents 50 μm. The luminal diameter (FIG. 1E) and mucosa height (FIG. 1F) increase as a function of culture length and passage number.

FIG. 2A-2L. Immunofluorescence microscopy analysis of equine airway organoid differentiation. Thoroughbred foal lung (FIG. 2A), P0 bronchioalveolar organoid (FIG. 2B) and P1 bronchioalveolar organoid (FIG. 2C) stained with markers of basal cells (KRT5, magenta), club cells (CCSP, red), and acetylated tubulin (Ac-Tub, green). (FIG. 2D) Thoroughbred foal lung, (FIG. 2E) P0 bronchioalveolar organoid, and (FIG. 2F) P3 organoid stained with markers of basal cells (KRT5, magenta), stem cells (SOX2, red), and goblet cells (MUCSAC, green). (FIG. 2G) Thoroughbred foal lung, (FIG. 2H) P0 bronchioalveolar organoid, and (FIG. 2I) P4 respiratory organoid stained with markers of basal cells (p63, green), club cells (CCSP, red), and AT2 cells (SPC, magenta). Representative hematoxylin and eosin stain demonstrating cuboidal and pseudostratified epithelium in the foal lung (FIG. 2J), P0 bronchioalveolar organoid (FIG. 2K), and P1 respiratory organoids (FIG. 2L). In 2A-2I, scale bar represents 20 μm.

FIG. 3A-3C. Development of an equine lung-on-a-chip. FIG. 3A: Equine lung basal cells seeded into the parenchymal channel of a flexible microfluidic chip rapidly form a monolayer on the chip membrane. Pulmonary endothelial cells seeded into the lower ‘vasculature’ channel form a robust barrier. Image depicts a section from the center of the microfluidic chip where endothelial and basal cells are interfaced. FIG. 3B: Magnification of the basal cell channel of the lung-chip depicted in (3A) three days post-seeding. Tight-junction barriers are observed between basal cells within the lung-chip. FIG. 3C: qPCR analysis of bronchioalveolar organoid cellular differentiation. Organoids grown in either expansion media (28 days) or expansion media (7 days) followed by transition to differentiation media for 21 days. Levels of stem cell (SOX2), basal cell (P63 and KRT5), epithelial cell (EpCAM), ciliated cell (FOXJ1), goblet cell (MUC5AC), and club cell (CCSP) markers are expressed relative to organoids maintained in expansion media. Scale bar represents 100 μm in 3A and 3B.

FIG. 4A-4F. Equine jejunal enteroids develop crypt-like structures and form a central lumen. FIG. 4A: Representative phase contrast microscopy of P0 equine jejunal organoids demonstrating rapid development of budding crypt-like structures. Images are presented to scale. FIG. 4A: Schematic of human enteroid development. Equine enteroids follow similar developmental kinetics. FIG. 4C-4F: High pass organoids develop cystic structures enriched in LGR5+ stem cells. Scale bar represents 50 μm in all panels. Schematic in 4B adapted from Date et al., 2015.

FIG. 5A-51I. Development of an equine intestine-on-a-chip. FIG. 5A: Schematic representation of equine intestine-on-a-chip, including the top view of the Emulate chip (left), and a vertical slice (right) showing the epithelial channel (1, blue), the microvasculature channel (2, pink), and microchannels populated by organoid-derived intestinal epithelial cells (3) and endothelial cells (4), separated by flexible, porous membrane coated by extracellular matrices (5). FIG. 5B: Brightfield image of an equine intestine-chip cultured for 12 days in the presence of fluid flow and stretch (30 μl/h flow, 10% strain, 0.2 Hz). FIG. 5C: High magnification, phase contrast image of the intestinal epithelium channel depicted in b. FIG. 5D: Phase contrast image of a vertical section of a mature equine intestine-chip demonstrating tissue architecture of the intestinal epithelium (upper channel). FIG. 5E: Intestinal epithelium height measured in situ over a 400 μm section of the intestine-chip depicted in b. FIG. 5F: Phase contrast image of confluent endothelial cells lining the bottom microvasculature channel of an equine intestine-chip. FIG. 5G: qPCR analysis of transcripts associated with intestine cell differentiation in organ-chips compared to 3D enteroid culture. (FIG. 5H: Testosterone conversion by epithelial CYP3A89 in equine intestine-chips was monitored by LC-MS/MS. Graph depicts the percent conversion of testosterone to 6-hydroxytestosterone at two concentrations. Scale bar represents 100 μm in panels 5B, 5C, 5D, and 5F. Schematic in 5A adapted and modified from Kasendra et al., eLife 2020.

FIG. 6A-61I. Equine liver organoids develop from hepatocytes and cholangiocytes. FIG. 6A: Representative phase contrast microscopy of P0 equine cholangiocyte organoids demonstrating typical spheroid morphology. FIG. 6B: Phase contrast image of a cholangiocyte organoid at P3, day 6. FIG. 6C: High magnification of the organoid presented in b. FIG. 6D: High pass (P7) cholangiocyte organoids maintain spherical morphology. FIG. 6E: Representative phase contrast image of an early pass hepatocyte organoid. Hepatocyte organoids form large, folded structures. FIGS. 6F and 6G: Higher magnification detail of the organoid presented in e, demonstrating the formation of tight junctions. FIG. 6H: High pass (P7) hepatic organoids maintain a large, budded morphology. Scale bar represents 50 μm in all panels

FIG. 7A-7F. Equine tubuloids derived from cortical kidney. FIGS. 7A and 7B: Representative phase contrast microscopy of P0, day 14 equine kidney organoids demonstrating typical spheroid morphology. FIG. 7C-7F: Phase contrast images of P0 differentiated kidney organoids 7 days after withdrawal of specific growth factors. Scale bar represents 20 μm in all panels.

FIG. 8A-8B. Equine hepatic organoid-derived models. FIG. 8A includes an image of cells dissociated from equine hepatic organoids, seeded onto a transwell apparatus, and cultured under static conditions to yield monolayers of differentiated cells by 12-days post seeding. FIG. 8B includes an image of cells dissociated from equine hepatic organoids, seeded onto an organ-chip microfluidic device, and cultured under continuous, directional fluid flow to drive cellular differentiation. The equine organ-on-a-chip microfluidic devices rapidly formed differentiated, three-dimensional structures that more closely resemble native liver tissue architecture within the 12-day time frame. Scale bar represents 100 μm.

FIG. 9A-9I. Equine proximal airway models of viral infection. Equine bronchioalveolar organoids were dissociated into single cell suspensions that were subsequently seeded and cultured to allow cellular differentiation. Monolayers were infected with either equine influenza virus A (EIV) or equine herpes virus 1 (EHV-1), or were mock infected to serve as a control. At 24 h and 48 h, infected and mock-infected transwells were imaged by phase contrast microscopy to evaluate viral-induced cytopathic effects. FIG. 9A is an image from a mock-infected transwell at 24 h. FIGS. 9B-9C are images from EIV-infected transwells at an MOI of 1 at 24 h. FIG. 9D is an image from a mock-infected transwell at 48 h. FIG. 9E is an image from an EIV-infected transwell at an MOI of 0.1 at 48 h. FIG. 9F is an image from an EIV-infected transwell at an MOI of 1 at 48 h. FIG. 9G is an image from a mock-infected transwell at 48 h. FIGS. 9H-9I are images from EHV-1-infected transwells at an MOI of 1 at 48 h. In all panels, scale bar represents 50 μm.

FIG. 10A-10E. Identification and quantification of Ciclesonide metabolites produced by equine airway and hepatic organoid-derived tissue systems. FIG. 10A includes the structure of Ciclesonide, which is a pro-drug that is metabolized in the lungs to the active form, desisobutyryl-Ciclesonide (des-CIC). FIG. 10B includes MS spectra showing representative fragmentation pattern of kinetic des-CIC conversion from CIC hydrolysis generated by equine bronchioalveolar organoid-derived transwells treated with pro-drug. FIGS. 10C-10D plot quantification of des-CIC metabolite produced by equine airway organoid-derived transwells treated with 50 μM (FIG. 10C) or 500 μM (FIG. 10D) Ciclesonide as a function of time. FIG. 10E plots quantification of des-CIC metabolite produced by equine hepatic organoid-derived transwells treated with 500 μM Ciclesonide as a function of time.

FIG. 11A-11D. Equine gastrointestinal tract organoids. Representative phase contrast microscopy images depicting the development of equine glandular stomach-derived gastric organoids (FIG. 11A), duodenal enteroids (FIG. 11B), mid-jejunal enteroids (FIG. 11C), and ileal enteroids (FIG. 11D). The scale bars in FIG. 11A-11D represents 50 μm.

FIG. 12A-12D. Expansion and differentiation of equine hepatic organoids. Hepatic organoids were derived from multiple thoroughbred donors and expanded via continual passage. FIG. 12A includes representative phase contrast microscopy (low magnification) image of equine hepatic organoids cultured in expansion media. Scale bar represents 100 μm. FIG. 12B includes a high magnification phase contrast microscopy image of undifferentiated hepatic organoids. Equine hepatic organoids can be terminally differentiated through withdrawal of specific growth factors at early (FIG. 12C, passage 4) or late (FIG. 12D, passage 14) passages. In FIG. 12B-12D, scale bar represents 50 μm.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes equine organoid systems that exhibit organ- and tissue-specific characteristics.

The engineered organoids of the presently-disclosed subject matter provide a renewable and reproducible platform that enables the study of equine-specific disease mechanisms and experimental concepts that are not currently feasible in vivo. For example, coupled to genomic manipulation technologies, organoids afford an ethical and biologically-relevant model system in which to perform studies designed to elucidate genetic susceptibility to disease. Organoids of the presently-disclosed subject matter also facilitate molecular-level experimentation that cannot be performed in the horse or in traditional two-dimensional and immortalized cell culture-based systems.

Additionally, owing to their virtually unlimited ability to self-renew and expand, organoids bridge the gap between the laboratory and disease models, thereby providing an attractive alternative to animal experimentation. For example, equine organoids of the presently-disclosed subject matter provide a relevant model system in which to identify and analyze therapeutic interventions prior to in vivo testing, leading to higher rates of success in the horse.

The equine organoid systems and methods as disclosed herein have utility in addressing outstanding questions, for example, in viral, bacterial, and parasitic infection biology; zoonotic disease transmission; drug discovery, toxicology, and pre-clinical therapeutic candidate analyses; allergic and immune-modulated conditions; tissue morphogenesis, architecture, and remodeling; and developmental biology.

Furthermore, mammalian genome editing technology can also be incorporated for purpose of ethically manipulating equine organoid systems (e.g., using CRISPR-Cas9). Such systems can be useful, for example, to identify specific host factors that contribute to disease susceptibility, organ-specific innate defense mechanisms, and infection control.

The presently-disclosed subject matter further includes integration of diverse organoid-derived tissues described herein into an innovative central system or ‘horse-on-a-chip’ microfluidic device for additional physiologic manipulation and biomechanical control.

Accordingly, disclosed herein are sustainable, biologically-relevant equine organoid systems that exhibit organ- and tissue-specific characteristics. Also disclosed herein are platforms that integrate one or more of the organoid systems into microfluidic organ-chip(s) that recapitulate tissue microarchitecture and incorporate the mechanical stress of an organ or tissue in a horse.

For example, disclosed herein is an equine airway organoid system that exhibits lung- and trachea-specific characteristics, including coordinated ciliated cell beating and mucus production. Of significant concern, equine influenza virus (EIV) and equine herpesvirus (EHV) are two of the most important and prevalent respiratory pathogens of the horse. Equine lung organoids and lung-on-a-chip microfluidic devices will provide a biomimetic system in which to study respiratory viral infection dynamics under near-physiologic conditions thus representing an attractive alternative to in vivo infection challenge models. In addition to viral pathogenesis, the airway organoid and respiratory chip systems can be used to explore mechanisms underlying respiratory colonization by important bacterial pathogens, including Rhodococcus equi.

For another example, disclosed herein is an equine gastrointestinal organoid system. A major threat to the equine industry is the impact of gastrointestinal infectious diseases.

Infections by diverse gastrointestinal pathogens can result in life-threatening enteritis and colitis. Despite the importance of equine enteric infectious disease, mechanisms governing bacterial pathogenesis in the horse remain incompletely defined. The ability to introduce peristaltic biomechanical forces and directional fluid flow in the intestine-on-a-chip technology will enable innovative in vitro microbial pathogenesis studies in a life-like system that emulates the equine gastrointestinal tract.

The presently-disclosed subject matter can be used to explore models of equine tissue plasticity and morphogenesis, allergic and immune-modulated disease, and for the discovery and development of novel therapeutics. The presently-disclosed subject matter, including the organoid-to-microfluidic chip pipeline as disclosed herein, is highly relevant in the context of equine precision medicine, enabling diverse studies in disease modeling, drug design and development, personalized treatment strategies, and regenerative medicine in the context of the dynamic physiology of the horse. Such studies can include pharmacology and toxicology studies focused on drug metabolism rates, identification and quantitation of tissue-specific drug metabolites, analysis of pro-drug conversion, and delineating dose-dependent tissue responses.

The presently disclosed subject matter includes a method of culturing cells, which involves: obtaining equine primary cells derived from, and culturing the equine primary cells to promote self-assembly, formation, and differentiation of one or more organoids. In some embodiments, such as embodiments in which the cells are for respiratory organoids, the cells can be cultured with an air-liquid interface system. In other embodiments, the cells can be cultured such that organoids are grown submerged in liquid embedded within the extracellular matrix.

In some embodiments, the equine primary cells are derived from equine tissue selected from the group consisting of lung, trachea, stomach, small intestine duodenal, small intestine ileal, liver, bile duct, kidney, bone, skin, pancreas, cecum, colon, brain, neuron, salivary gland, retina, placenta, uterus, and mammary gland. In some embodiments, the equine primary cells are further derived from equine small intestine jejunal tissue.

In some embodiments, the equine primary cells are derived from equine tissue including lung and/or trachea. In some embodiments, the equine primary cells are derived from equine tissue including glandular stomach, non-glandular stomach, and/or bile duct cholangiocyte. In some embodiments, the equine primary cells are derived from equine tissue including small intestine duodenal, small intestine jejunal tissue, and/or small intestine ileal. In some embodiments, the equine primary cells are derived from equine tissue including liver hepatocyte. In some embodiments, the equine primary cells are derived from equine tissue including kidney epithelial tubuloid. In some embodiments, the equine primary cells are derived from equine tissue including bone. In some embodiments, the equine primary cells are derived from equine tissue including skin. In some embodiments, the equine primary cells are derived from equine tissue including brain. In some embodiments, the equine primary cells are derived from equine tissue including salivary gland. In some embodiments, the equine primary cells are derived from equine tissue including retina. In some embodiments, the equine primary cells are derived from equine tissue including placenta and/or uterus. In some embodiments, the equine primary cells are derived from equine tissue including mammary gland.

In some embodiments, the method of culturing cells also involves dissociating the organoid for expansion and passage.

In some embodiments, the method of culturing cells also involves co-culturing the organ-derived stem cells with equine endothelial cells, which can mimic the presence of native vasculature and produce growth factors for stimulating organoid differentiation.

In some embodiments, the method of culturing cells also involves co-culturing the organ-derived stem cells with equine primary cells isolated from whole blood. In some embodiments, the primary cells comprise neutrophils and/or macrophages.

The method of culturing cells can additionally involve use of equine tissue obtained from multiple horses, which can provide a number of benefits as will be understood by the skilled artisan.

In some embodiments, the multiple horses include horses of the same breed. For example, in some embodiments, the multiple horses can be two or more horses of a single breed selected from the group that includes, but is not limited to: Thoroughbred, Quarter Horse, Warmblood, Standardbred, Saddlebred, and mixed breed. In some embodiments, the multiple horses include horses of distinct breeds. For example, in some embodiments, the distinct breeds can be two or more breeds selected from the group that includes, but is not limited to: Thoroughbred, Quarter Horse, Warmblood, Standardbred, Saddlebred, and mixed breed.

In some embodiments, the multiple horses include horses of the same sex. For example, in some embodiments, the multiple horses can be two or more horses that are male. In some embodiments, the multiple horses can be two or more horses that are female. In some embodiments, the multiple horses include horses of distinct sexes such that the multiple horses include horses include male and female horses.

In some embodiments, the multiple horses includes horses that are healthy. In some embodiments, the multiple horses includes horses include horses having a condition or disease. In some embodiments, the multiple horses can have distinct conditions or diseases, and in some embodiments, the multiple horses all have the same particular condition or disease. In some embodiments, the multiple horses includes horses having a condition with a documented genetic component. In some embodiments, the multiple horses includes horses having recurrent airway obstruction (RAO). In some embodiments, the multiple horses includes horses having hyperkalemic periodic paralysis (HYPP).

In some embodiments, the method of culturing cells also involves transfecting the cells with a vector including a nucleotide encoding a polypeptide comprising a luminescent protein. In some embodiments, the vector further includes a nucleotide encoding a constitutively active or expressed protein, or an inducible protein that can be controlled, by methods known to those of ordinary skill in the art. The luminescent protein can be, for example, a bioluminescent protein or a fluorescent protein. In some embodiments, the fluorescent protein can be, for example, green fluorescent protein, yellow fluorescent protein, blue fluorescent protein, cyan fluorescent protein, a monomeric red fluorescent protein such as mCherry, or a superfolder fluorescent protein. In some embodiments, the bioluminescent protein can be a luciferase, such as a luciferase from fire fly. In some embodiments, the polypeptide is a fusion protein comprising the luminescent protein and a marker protein. In some embodiments, the marker protein is a stem cell marker. For example, the stem cell marker could be a leucine-rich repeat-containing G-protein coupled receptor 5 (LGR5). In some embodiments, the method also involves monitoring and sorting cells based on luminescence.

The presently disclosed subject matter includes a method of culturing cells to obtain one or more organoids, as disclosed herein, and further comprising transplanting the one or more organoids into a horse. The presently disclosed subject matter includes a method of treating a horse, which involves transplanting one or more organoids into the horse.

The presently disclosed subject matter includes a method of preparing an in vitro equine organ model system. In some embodiments, the method of preparing an in vitro equine organ model system involves providing a microfluidic device comprising an upper chamber and a lower chamber, separated by a membrane that permits the exchange of cellular signals and soluble molecules; dissociating a three-dimensional organoid comprising multiple cell types, wherein the organoid was prepared using equine tissue associated with an organ of interest; selecting equine primary cells from the dissociated organoid; seeding the upper chamber of the device with the equine primary cells; expanding the equine primary cells in a submerged two-dimensional adherent cell culture; and differentiating the equine primary cells to create differentiated cell types associated with the organ of interest. Beneficially, the method can be used without the need for cell sorting, and thus does not need to make use of an antibody for cell sorting.

In some embodiments, the method of preparing the in vitro equine organ model system also involves seeding the lower chamber of the microfluidic device with equine endothelial cells.

In some embodiments, the method of preparing the in vitro equine organ model system also involves expanding the equine primary cells further comprises applying fluid flow, thereby initiating differentiation of the equine primary cells. In some embodiments of the method, the organ(s) of interest is from the airway, and further comprising applying air flow and mechanical movement to obtain to obtain a pseudostratified epithelium. In some embodiments of the method, the organ(s) of interest is from the intestine, and further comprising continued application of fluid flow and applying mechanical movement to obtain a pseudostratified epithelium. In some embodiments of the method, the organ(s) of interest is from the liver, and further comprising continued application of fluid flow to obtain a pseudostratified epithelium. In some embodiments of the method, the organ(s) of interest is from the kidney, and further comprising continued application of fluid flow to obtain a pseudostratified epithelium.

In some embodiments of the method, the equine primary cells are from a tissue or organoid selected from the group consisting of lung, trachea, stomach, intestine, liver, bile duct, kidney, bone, skin, pancreas, cecum, colon, brain, neuron, salivary gland, retina, placenta, uterus, and mammary gland.

In some embodiments of the method, the organ of interest comprises one or more respiratory system organs. In some embodiments, the one or more respiratory system organs comprise lung and/or trachea. In some embodiments of the method, the organ of interest comprises one or more gastrointestinal tract organs. In some embodiments, the one or more gastrointestinal tract organs comprise stomach and/or small intestine. In some embodiments of the method, the organ of interest comprises one or more hepatic system organs. In some embodiments, the one or more hepatic system organs comprise liver and/or bile duct. In some embodiments of the method, the organ of interest comprises one or more renal (urinary) system organs. In some embodiments, the one or more renal system organs comprise kidney.

In some embodiments, the method of preparing the in vitro equine organ model system also involves seeding the upper chamber of the microfluidic device with stem cells of the organ(s) of interest.

In some embodiments of the method of preparing the in vitro equine organ model system, the equine primary cells are stem cells. In some embodiments, the stem cells are airway stem cells. In some embodiments, the airway stem cells are bronchioalveolar basal cells. In some embodiments, the stem cells are intestinal stem cells. In some embodiments, the stem cells are hepatic stem cells. In some embodiments, the stem cells are renal stem cells.

The method of preparing the in vitro equine organ model system can additionally involve use of equine tissue obtained from multiple horses, which can provide a number of benefits as will be understood by the skilled artisan.

In some embodiments, the multiple horses include horses of the same breed. For example, in some embodiments, the multiple horses can be two or more horses of a single breed selected from the group that includes, but is not limited to: Thoroughbred, Quarter Horse, Warmblood, Standardbred, Saddlebred, and mixed breed. In some embodiments, the multiple horses include horses of distinct breeds. For example, in some embodiments, the distinct breeds can be two or more breeds selected from the group that includes, but is not limited to: Thoroughbred, Quarter Horse, Warmblood, Standardbred, Saddlebred, and mixed breed.

In some embodiments, the multiple horses include horses of the same sex. For example, in some embodiments, the multiple horses can be two or more horses that are male. In some embodiments, the multiple horses can be two or more horses that are female. In some embodiments, the multiple horses include horses of distinct sexes such that the multiple horses include horses include male and female horses.

In some embodiments, the multiple horses includes horses that are healthy. In some embodiments, the multiple horses includes horses include horses having a condition or disease. In some embodiments, the multiple horses can have distinct conditions or diseases, and in some embodiments, the multiple horses all have the same particular condition or disease. In some embodiments, the multiple horses includes horses having a condition with a documented genetic component. In some embodiments, the multiple horses includes horses having recurrent airway obstruction (RAO). In some embodiments, the multiple horses includes horses having hyperkalemic periodic paralysis (HYPP).

The presently-disclosed subject matter further includes an in vitro equine organ model system, prepared according to any of the methods as disclosed herein.

The presently-disclosed subject matter further includes a kit for an in vitro equine organ model system. In some embodiments, the kit can include equine primary cells, wherein the equine primary cells are derived from equine tissue associated with an organ of interest, or derived from an organoid prepared using equine tissue associated with the organ of interest.

In some embodiments of the kit for an in vitro equine organ model system includes an organoid prepared using equine tissue associated with the organ of interest. In some embodiments, the organoid is selected from the group consisting of bronchioalveolar organoid, tracheosphere, hepatic organoid, kidney tubuloid, gastric organoid, duodenal enteroid, jejunal enteroid, and ileal enteroid. In some embodiments, the organoid is selected from the group consisting of UKEOP0001, UKEOP0002, UKEOP0003, UKEOP0004, UKEOP0005, UKEOP0006, UKEOP0007, UKEOP0008, UKEOP0009, UKEOP0010, UKEOP0011, UKEOP0012, and UKEOP0013.

In some embodiments, the kit can additionally include regents culturing the equine primary cells. In some embodiments, the reagents comprise components for expanding and/or differentiating the cells. In some embodiments, the reagents comprise cell culture media. In some embodiments, the reagents further comprise growth factors specific to a tissue microenvironment of the organ of interest.

Some embodiments of the kit include a microfluidic device, comprising an upper chamber and a lower chamber, separated by a membrane that permits the exchange of cellular signals and soluble molecules. In some embodiments, the kit includes a culture apparatus containing basement membrane matrix.

Some embodiments of the kit also include equine endothelial cells. In some embodiments, the kit includes stem cells of an organ of interest.

In some embodiments of the kit for an in vitro equine organ model system, the equine primary cells are from a tissue or organoid selected from the group consisting of lung, trachea, glandular stomach, small intestine duodenal, small intestine jejunal, small intestine ileal, liver hepatocyte, bile duct cholangiocyte, and kidney epithelial tubuloid.

In some embodiments of the kit for an in vitro equine organ model system, the organ of interest is selected from the group consisting of: lung, trachea, stomach, intestine, liver, bile duct, kidney, bone, skin, pancreas, cecum, colon, brain, neuron, salivary gland, retina, placenta, uterus, and mammary gland. In some embodiments of the kit, the organ of interest comprises one or more respiratory system organs. In some embodiments, the one or more respiratory system organs comprise lung and/or trachea. In some embodiments of the kit, the organ of interest comprises one or more gastrointestinal tract organs. In some embodiments, the one or more gastrointestinal tract organs comprise stomach and/or small intestine. In some embodiments of the kit, the organ of interest comprises one or more hepatic system organs. In some embodiments, the one or more hepatic system organs comprise liver and/or bile duct. In some embodiments of the kit, the organ of interest comprises one or more renal (urinary) system organs. In some embodiments, the one or more renal system organs comprise kidney.

In some embodiments of the kit for an in vitro equine organ model system, the equine primary cells are stem cells. In some embodiments of the kit, the stem cells are airway basal epithelial cells. In some embodiments, the airway basal epithelial cells are bronchioalveolar basal cells. In some embodiments of the kit, the stem cells are intestinal stem cells. In some embodiments of the kit, the stem cells are hepatic stem cells. In some embodiments of the kit, the stem cells are renal stem cells.

The kit for an in vitro equine organ model system can include cells derived from organoids prepared from or cells derived from equine tissue obtained from multiple horses, which can provide a number of benefits as will be understood by the skilled artisan.

In some embodiments, the multiple horses include horses of the same breed. For example, in some embodiments, the multiple horses can be two or more horses of a single breed selected from the group that includes, but is not limited to: Thoroughbred, Quarter Horse, Warmblood, Standardbred, Saddlebred, and mixed breed. In some embodiments, the multiple horses include horses of distinct breeds. For example, in some embodiments, the distinct breeds can be two or more breeds selected from the group that includes, but is not limited to: Thoroughbred, Quarter Horse, Warmblood, Standardbred, Saddlebred, and mixed breed.

In some embodiments, the multiple horses include horses of the same sex. For example, in some embodiments, the multiple horses can be two or more horses that are male. In some embodiments, the multiple horses can be two or more horses that are female. In some embodiments, the multiple horses include horses of distinct sexes such that the multiple horses include horses include male and female horses.

In some embodiments, the multiple horses includes horses that are healthy. In some embodiments, the multiple horses includes horses include horses having a condition or disease. In some embodiments, the multiple horses can have distinct conditions or diseases, and in some embodiments, the multiple horses all have the same particular condition or disease. In some embodiments, the multiple horses includes horses having a condition with a documented genetic component. In some embodiments, the multiple horses includes horses having recurrent airway obstruction (RAO). In some embodiments, the multiple horses includes horses having hyperkalemic periodic paralysis (HYPP).

The presently-disclosed subject matter also includes a method of predicting results of administration of a sample to a horse, which involves contacting the sample to an in vitro equine organ model system as disclosed herein. In some embodiments, the sample comprises a drug of interest. In some embodiments, the method also involves obtaining data. In some embodiments, the method also involves providing a customer report containing the obtained data.

In some embodiments, the data obtained can be drug toxicity data, which could include, for example, tissue-specific and metabolite/intermediate-specific toxicity. In some embodiments, the method also involves obtaining data related to drug pharmacokinetics. In some embodiments, the method also involves obtaining information regarding equine-specific metabolites of a drug, which is particularly useful given that tissue specific can be considered, and the method provides for a germ-free environment, as compared to studies, e.g., in a live animal. In some embodiments, the method also involves obtaining data regarding tissue-specific efficacy of a drug, tissue-specific prodrug conversion of a drug, and/or target-tissue-specific metabolism of a drug, e.g., information regarding which enzyme and which tissue is metabolizing a drug. In some embodiments, the method also involves obtaining data regarding efficacy of a drug for specific infection models and/or efficacy of microbiome on drug metabolism. In some embodiments, the method also involves obtaining data screening efficacy of an existing drug for use in a horse, for example, a drug that was rejected for use in humans, but could prove effective in horses.

In some embodiments of the method, the in vitro equine organ model system is for a first organ of interest, and the method also involves collecting metabolites from the system for the first organ of issue and applying the collected metabolites to a second in vitro equine organ model system for a second organ of interest. In some embodiments, the first organ of interest is liver.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Respiratory Organoids

A renewable in vitro equine system was prepared for use in studying respiratory infection models, as described herein. Methods were developed to culture and differentiate equine-derived tracheal and distal lung stem cells into 3D organoids.

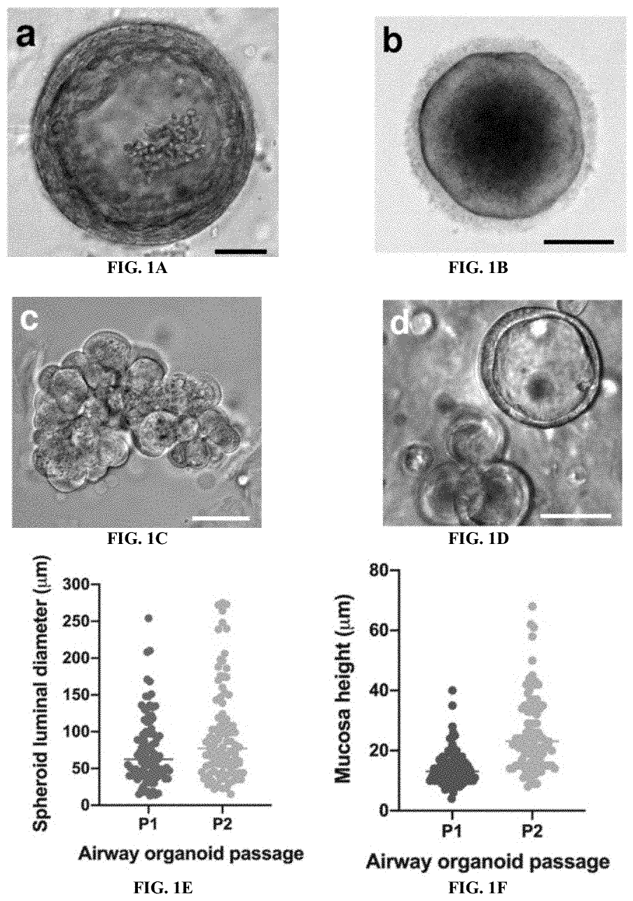

Lungs and trachea were dissected from a thoroughbred foal euthanized for reasons unrelated to the current study. With consideration to protocols developed for human and mouse lung organoid culture, a 3D air-liquid interface system containing a defined murine basement membrane matrix was established and used to promote tracheosphere and distal lung airway organoid formation. (FIG. 1A-1F).

FIG. 1A-1C include representative images of UKEOP0001 and FIG. 1D includes a representative image of UKEOP0002. FIG. 1E-1F include data obtained from UKEOP0001.

Equine tracheospheres and bronchioalveolar organoids rapidly self-assembled and formed within 5 days of air-liquid interface culture, with proximal lung organoids differentiating within approximately 4-6 weeks in vitro (FIG. 1A-1D). Equine tracheospheres exhibited characteristics of native trachea, including robust mucus production and ciliated cells that coordinated to beat the mucus in a defined pattern within the tracheosphere lumen.

At 21-days post-seeding, airway organoids [designated passage 0 (P0)] were dissociated for expansion and passage into new air-liquid interface cultures [designated P1 (FIG. 1E-1F)]. Second (P1) and third generation (P2) tracheospheres and proximal airway organoids self-organized and formed mature spheroids within one week of culture. The maximal diameter of passaged organoids increased over time, and continued to increase over four weeks (FIG. 1E-1F).

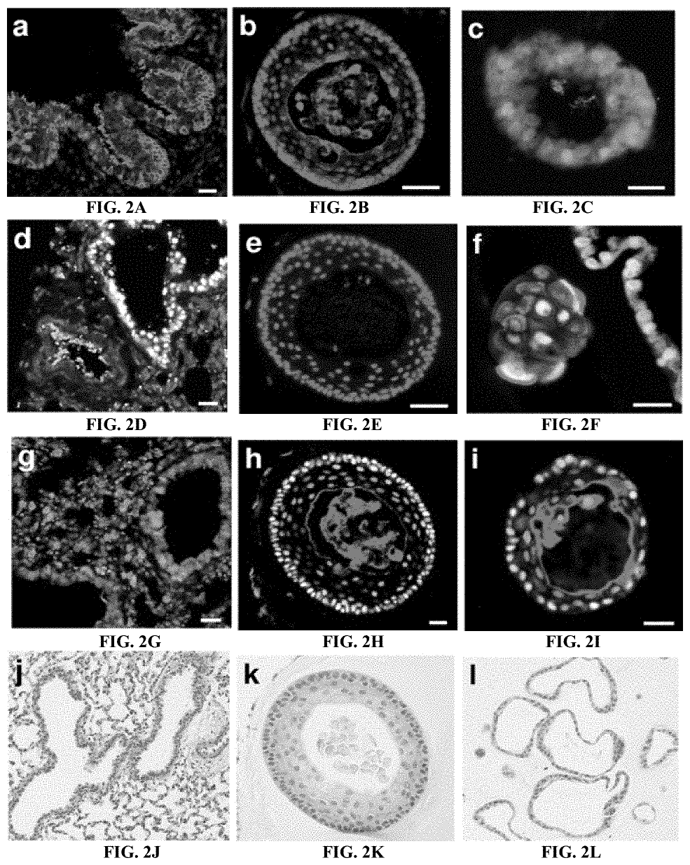

Representative wells from each organoid passage were formalin fixed and histopathology analysis was conducted. Histochemical and immunohistochemical (FIG. 2A-2L) analysis of embedded respiratory organoids revealed pseudostratified epithelia with cuboidal and columnar cells, diffuse cytokeratin staining, with mucus frequently observed in the organoid interior. In addition, ciliated cells were commonly localized to the apical surface of tracheosphere lumens (data not shown). FIG. 2A-2L are images from UKEOP0001, but are representative of and related to others that were similarly prepared, such as UKEOP0002.

In addition to generating trachea-derived organoids (FIG. 1D), methods were also developed to co-culture equine proximal lung basal cells with endothelial ‘support cells’ that mimic the presence of native vasculature and produce growth factors that stimulate organoid differentiation.

In these studies, dissociated lung tissue was seeded with equine endothelial cells derived from a thoroughbred foal. Under these conditions, bronchioalveolar organoids developed and matured within 4-6 weeks of air-liquid interface culture (FIG. 1A-1D and FIG. 2A-2L).

Occasionally, bronchioalveolar organoids differentiated in a ‘apical-out’ orientation (FIG. 1B), producing ciliated cells surrounding the periphery of the organoid interfacing with the extracellular matrix. Multiple structures associated with differentiated bronchioalveolar organoids were observed, with the majority of organoids exhibiting a round, hollow morphology (expected ‘basal-out’ orientation) with thickened walls (FIG. 1A). Proximal airway organoids matured into large spheres with organoid diameters reaching nearly 2 mm in size (FIG. 1E-1F). The largest observed lung organoid displayed a surface area of 10.1 mm² and a volume of 3 mm³ (diameter 1.8 mm).

In addition to rounded morphologies, the formation of branched bronchioalveolar organoids (FIG. 1C) that exhibited air sac-like clusters were also observed.

Immunofluorescence microscopy of whole-mount proximal lung organoids revealed densely-packed nuclei and diffuse actin cytoskeleton throughout the organoid, as well as the presence of basal cells [cytokeratin 5 (KRT5) and p63, FIG. 2] and bronchioalveolar stem cells [SOX2, FIG. 2E-2F], club cells [club cell secretory protein (CCSP), FIG. 2D, 2I], goblet cells [mucin 5AC (MUC5ac), FIG. 2F], ciliated cells [Acetylated α-tubulin (Ac-TUB), FIG. 2C], and pulmonary surfactant-producing alveolar type II (AT2) pneumocytes [surfactant protein C (SPC), FIG. 2H-2I].

Bronchioalveolar organoids consistently developed and differentiated over multiple passages (FIG. 2), and respiratory organoid cultures were successfully expanded and maintained for >8 months.

Together, these studies demonstrate the ability to generate multiple lineages of differentiated equine respiratory tract organoids that provide the technological foundation for a variety of investigations in infectious disease, developmental biology, tissue plasticity and architecture, and drug discovery and toxicology.

### Example 2: Lung-On-a-Chip

An in vitro airway system was developed, which incorporates biomechanical forces to simulate breathing, as well as directional air and fluid flow across a multi-tissue, lung-like system.

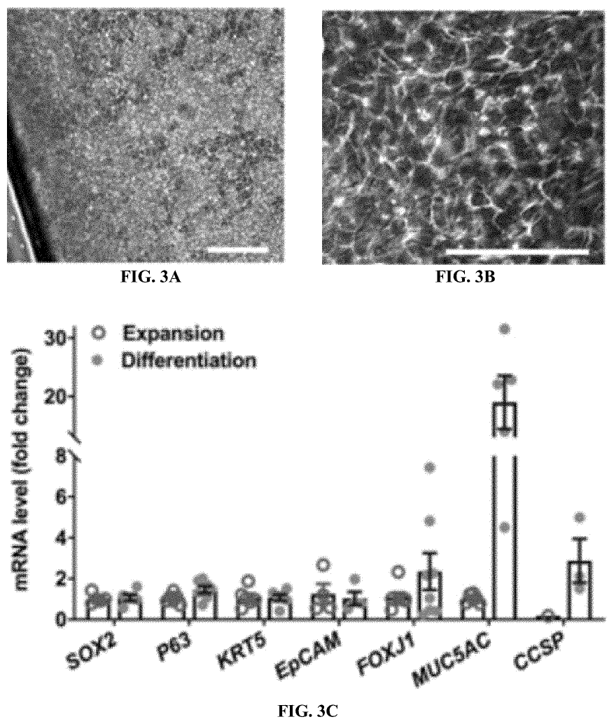

For these studies, a flexible microfluidic chip was used, which consists of two hollow channels—an upper parenchymal chamber seeded by respiratory basal cells and bronchioalveolar stem cells (BASCs) that give rise to the differentiated lung epithelium, and a lower vasculature channel lined with equine pulmonary endothelial cells (FIG. 3) separated by a membrane that permits the exchange of cellular signals and soluble molecules at the tissue-tissue interface.

FIG. 3A-3B include representative images of airway-chips derived from UKEOP0001. FIG. 3C includes data derived from 3D culture of UKEOP0001.

Methods were developed to transition the 3D bronchioalveolar organoids into two-dimensional (2D) monolayers enriched in basal cells and BASCs.

The enriched respiratory stem cell population was incorporated into the upper chamber of the organ-chip, and pulmonary endothelial cells were seeded into the lower chamber to form a multi-tissue interface (FIG. 3A).

Basal cells rapidly formed tight junctions (FIG. 3B) when mechanical stretch (simulating breathing) was applied in conjunction with transitioning of the upper channel from fluid flow to air flow. In this model, the epithelium is expected to differentiate within 21 days of transition to an air-liquid interface (ALI).

In addition, 3D organoid culture methods were developed to enhance cell differentiation (FIG. 3C). Using qPCR, it was determined that withdrawing specific chemical inhibitors and cellular growth factors from bronchioalveolar spheroid culture results in an increase in the expression of cell markers associated with ciliated cells (FOXJ1), goblet cells (MUC5AC), and club cells (CCSP) (FIG. 3C).

It is noted that, while introducing immune cells into organoid and organ-chip systems represents a significant challenge in the organoid systems, methods have been developed to induce the formation of ‘apical-out’ airway organoids, and the respiratory organoid systems have been translated into lung-on-a-chip devices, in which the upper ‘epithelial’ channel is separated from the lower ‘vasculature’ channel by a membrane consisting of defined pores that are of sufficient size for alveolar macrophage transmigration between channels.

The ability of neutrophils and macrophages to transmigrate from the vasculature channel into the epithelial channel of human lung-chip systems has been demonstrated, and it is contemplated that equine immune cells will transmigrate in response to epithelial pathogen challenge in the lung-chip. Furthermore, equine respiratory organoids can be translated to two-dimensional transwell systems in which immune cells can be directly added to the appropriate assay chamber in infection models.

Studies will also be conducted to evaluate cellular differentiation and function of equine lung-on-a-chip systems compared to bronchioalveolar organoid models.

### Example 3: Intestinal Organoids

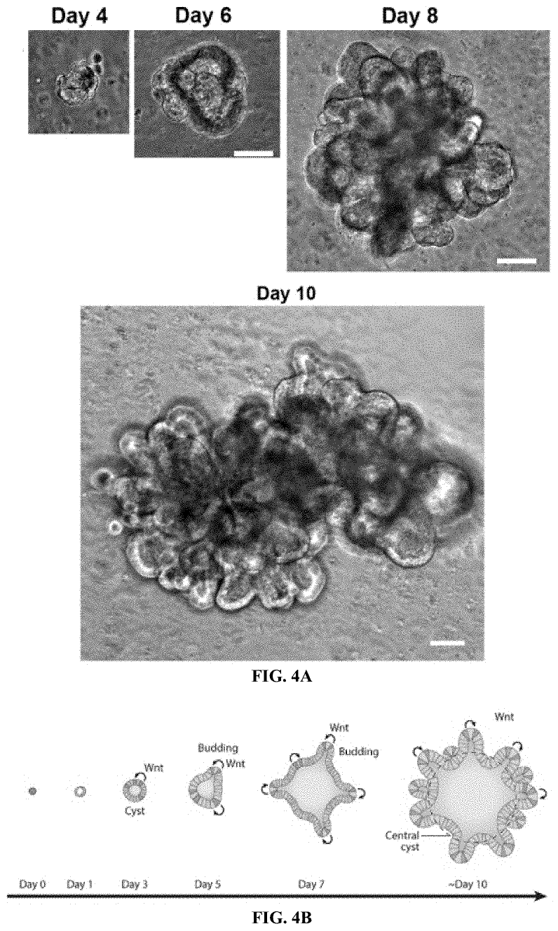

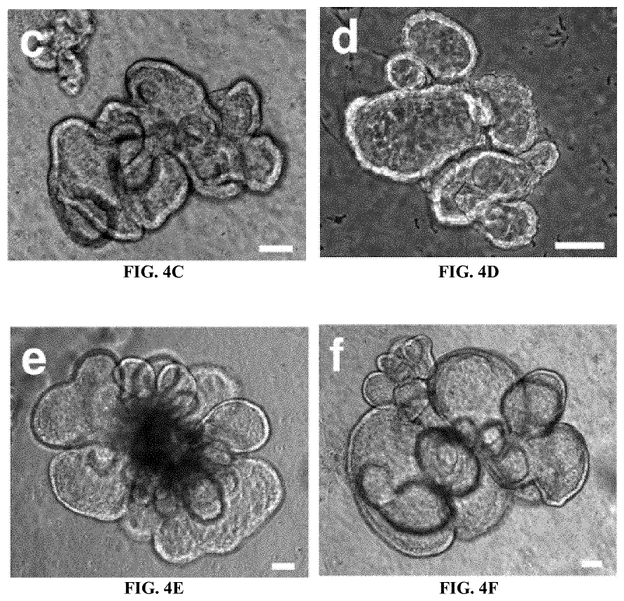

In addition to equine airway organoids, methods were designed and optimized to generate intestinal enteroids originating from the mid-jejunum of multiple thoroughbreds and mixed breed horses. FIG. 4A includes images from UKEOP0010, but is representative of and related to others that were similarly prepared, such as UKEOP0007, UKEOP0008, UKEOP0009, UKEOP0011, UKEOP0012, and UKEOP0013.

Enteroids arising from LGR5+ stem cells within isolated intestinal crypts rapidly formed budded organoids exhibiting a pseudolumen and multiple crypt-like structures (FIG. 4A) following the expected developmental kinetics observed for human intestinal organoids (FIG. 4B, which is a portion of FIG. 3 from Date and Sato²⁷).

By day 10, jejunal enteroids exhibited dark, necrotic cores consisting of shed mature enterocytes. At high passage (>P10) in optimized growth medium, equine enteroid morphology shifted to a predominately cystic phenotype (FIG. 4C-4F), as a result of LGR5 stem cell enrichment over the course of organoid expansion. FIG. 4C-4F includes images from UKEOP0010, but is representative of and related to others that were similarly prepared, such as UKEOP0007, UKEOP0008, UKEOP0009, UKEOP0011, UKEOP0012, and UKEOP0013.

In subsequent studies, methods were designed to generate equine enteroids originating from the glandular stomach, duodenum, jejunum, and ileum of thoroughbreds and mixed breed horses (data not shown).

It is noted that these studies and results are distinct from previously reported generation of equine enteroids derived from the mid-jejunum. In addition to jejunal organoids, organoids originating from the glandular stomach, duodenum, and ileum have been successfully cultured from mixed breed and thoroughbred donors. In addition, methods have been developed to translate equine intestinal organoids into a multi-tissue intestine-on-a-chip system that incorporates unilateral media flow, shear stress, and peristalsis-like biomechanical forces, thus representing a significant advance over previously published studies. Equine intestine-chips were also demonstrated to faithfully recapitulate differentiated tissue systems compared to proliferating intestinal organoids, thereby providing a more biologically-relevant system in which to study various aspects of intestinal and infection biology.

### Example 4: Intestine-On-a-Chip

In 3D organoid model systems pathogens are unable to reach the lumen of the structure where cellular receptors are expressed on the apical surface. Thus, an equine small intestine-on-a-chip was developed, which could be used to develop infection challenge models in which to study gastrointestinal (GI) viral and bacterial pathogenesis.

These intestinal organoid studies rely on the derivation of intestinal crypt stem cell populations (LGR5+ cells) that give rise to the differentiated intestinal epithelium. The goal of this exemplary small intestine-on-a-chip is to mimic the intestinal lumen; thus, focuses on generating LGR5-derived organoid populations to integrate into the intestine-chip technology, rather than growing cells from all layers of the intestine.

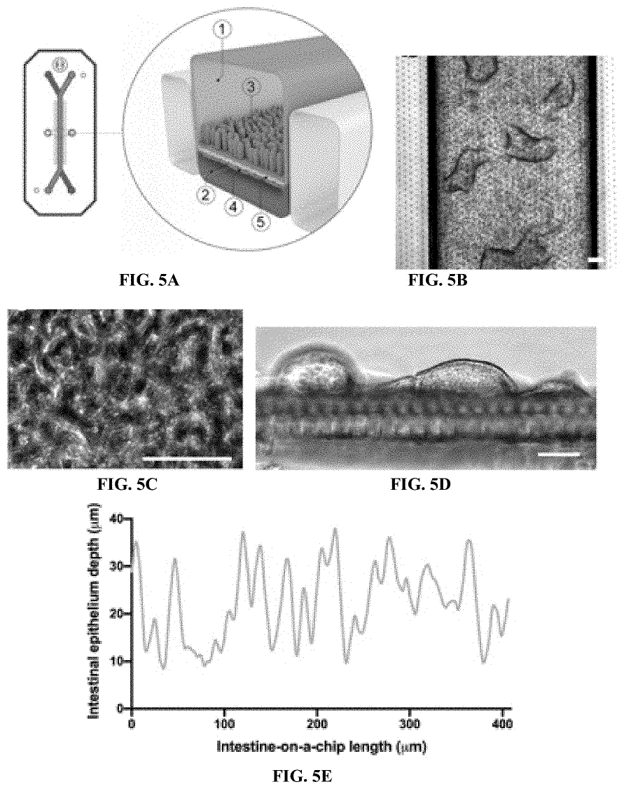

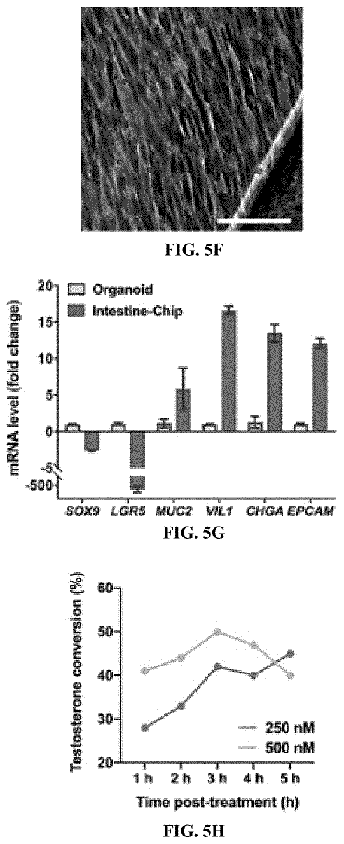

Methods were developed to seed jejunal enteroid fragments into microfluidic chips (FIG. 5A, which is a portion of FIG. 1 from Kasendra, et al.²⁸) to generate an equine small-intestine-on-a-chip. LGR5+ enteroid fragments rapidly formed a dense monolayer on in the chip upper channel (FIG. 5B) that differentiated into intestinal epithelial cells (FIGS. 5C and 5G) within 8-12 days under fluid flow and mechanical stretch to simulate peristalsis.

Equine endothelial cells lining the microvasculature channel (FIG. 5F) formed a confluent monolayer at the tissue-tissue interface. Intestinal epithelial cell differentiation in the organ-chip was verified via qPCR analysis and comparison to 3D enteroids cultured over the same time frame (FIG. 5G).

Compared to jejunal enteroids, markers of crypt stem cells (SOX9 and LGR5) were significantly decreased in the organ-chip, while expression of genes associated with goblet cells (MUC2), enteroendocrine cells (CHGA), enterocytes (VIL1), and epithelial cells (EPCAM) was significantly increased in the organ-chip (FIG. 5G).

Finally, studies were performed to evaluate the suitability of intestine chips for drug metabolism and CYP3A89 (CYP3A4 homolog (24)) induction. For these experiments, we inoculated testosterone into the inlet reservoir for the epithelial channel, flowed the testosterone-containing media through the chip at a rate of 30 μl/h, and collected samples from the effluent chambers from both the epithelial and vasculature channels. Liquid chromatography-mass spectrometry (LC-MS/MS) analysis of the effluent samples revealed CYP3A89-mediated conversion of testosterone to 6-hydroxytestosterone by 1 h post-treatment, with testosterone metabolism peaking by roughly 3 h (FIG. 5H).

FIGS. 5B-5E and 5G-5H includes images of or data obtain from UKEOP0010, but are representative of and related to others that were similarly prepared, such as UKEOP0007, UKEOP0008, UKEOP0009, UKEOP0011, UKEOP0012, and UKEOP0013.

Importantly, testosterone metabolites were detected only in the epithelial channel effluent, demonstrating intestine-chip barrier formation, and tissue- and channel-specific metabolic activity and CYP3A89 induction.

Reference is also made to FIG. 11A-11D, which includes a series of images of equine gastrointestinal tract organoids. FIG. 11A includes a representative phase contrast microscopy image depicting the development of UKEOP0006 equine glandular stomach-derived gastric organoids.

FIG. 11B includes a phase contrast microscopy image depicting the development of UKEOP0007 duodenal enteroids, which is representative of and related to others that were similarly prepared, such as UKEOP0008, UKEOP0009, UKEOP0010, UKEOP0011, UKEOP0012, and UKEOP0013.

FIG. 11C includes a phase contrast microscopy images depicting the development of UKEOP0011 mid-jejunal enteroids, which is representative of and related to others that were similarly prepared, such as UKEOP0007, UKEOP0008, UKEOP0009, UKEOP0010, UKEOP0012, and UKEOP0013.

FIG. 11D includes a phase contrast microscopy image depicting the development of UKEOP0013 ileal enteroids, which is representative of and related to others that were similarly prepared, such as UKEOP0007, UKEOP0008, UKEOP0009, UKEOP0010, UKEOP0011, and UKEOP0012.

Together, these studies demonstrate the ability to generate multi-tissue, microfluidic equine organ-chips.

### Example 5: Hepatic and Renal Organoids, and Organoid-Derived Models

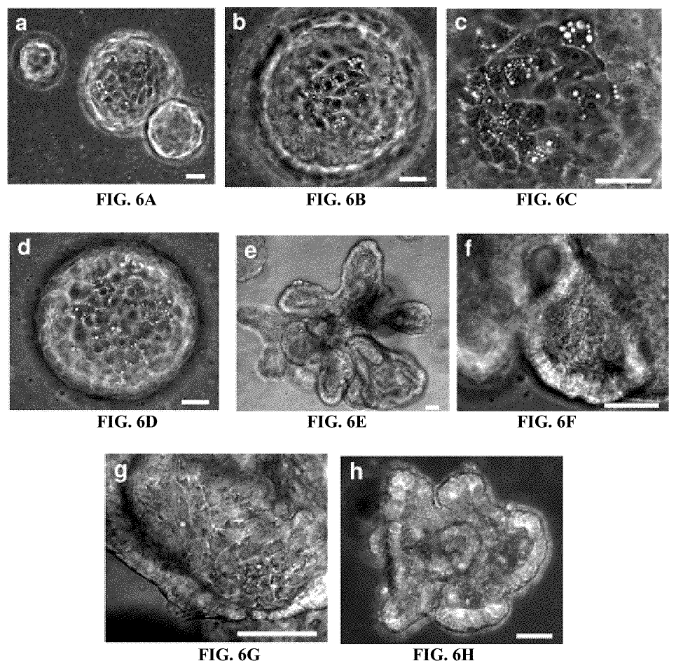

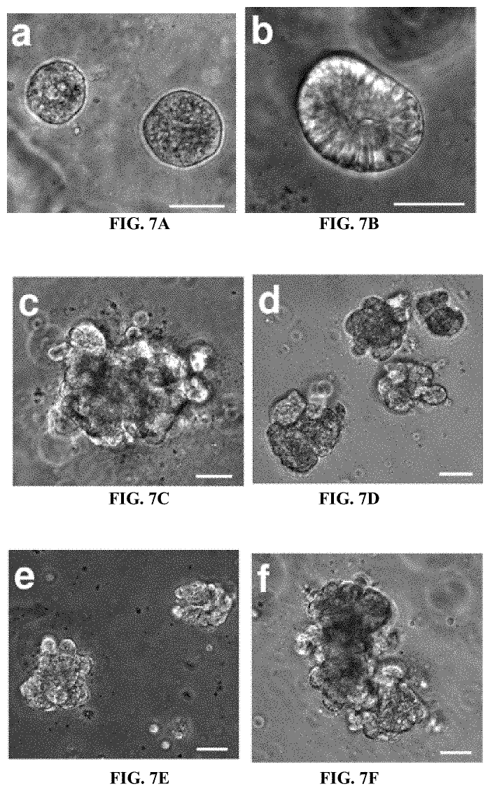

Methods were developed to generate hepatic and renal organoids derived from horse, e.g., thoroughbred, donor tissue. Equine liver organoids (both hepatocyte organoids and biliary epithelial-derived cholangiocyte organoids, FIG. 6), as well as kidney organoids (FIG. 7), were successfully generated.

Equine cholangiocyte organoids formed large spheroids with hollow lumens (FIG. 6A-6D) while hepatic organoids (FIG. 6E-6H) formed compact, folded structures. Liver organoids expanded to a diameter of several hundred microns within 7 days, and could be serially passaged via mechanical disruption every 7-10 days. FIG. 6A-6H includes images from UKEOP0003, but are representative of and related to others that were similarly prepared, such as UKEOP0004.

In contrast, with reference to FIG. 7A-7B, cortical kidney tubular fragments gave rise to small, dense spherical equine kidney tubuloids within 14 days. Methods were developed to differentiate kidney tubuloids through withdrawal of specific growth factors, resulting in a morphological shift from cystic organoids to budded organoids (FIG. 7C-7F). FIG. 7A-7B include images from UKEOP0005. FIG. 7C-7F also include images from UKEOP0005, but they are terminally differentiated.

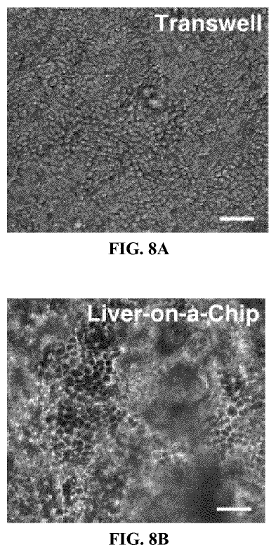

With reference to FIG. 8A-8B, equine hepatic organoids were dissociated into single cells. These cells were subsequently seeded onto a 0.4 μm pore transwell apparatus (FIG. 8A) or an organ-chip microfluidic device (FIG. 8B). Stem cells were cultured under either static conditions (transwell), or under continuous, directional fluid flow (liver-on-a-chip) to drive cellular differentiation. Compared to transwell monolayers that displayed differentiated cells by 12 days post-seeding (FIG. 8A), equine liver-on-a-chip microfluidic devices (FIG. 8B) rapidly formed differentiated, three-dimensional structures that more closely resemble native liver tissue architecture within the same time frame. FIG. 8A-8B include images from UKEOP0003, but are representative of and related to others that were similarly prepared, such as UKEOP0004.

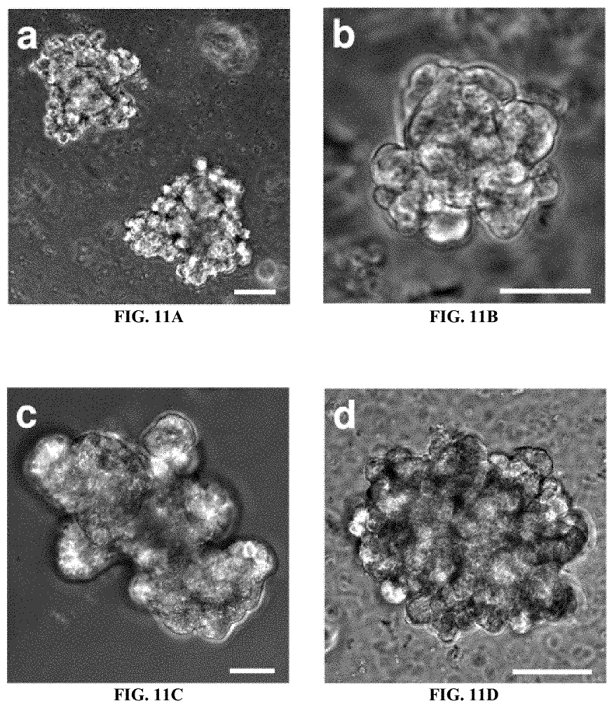

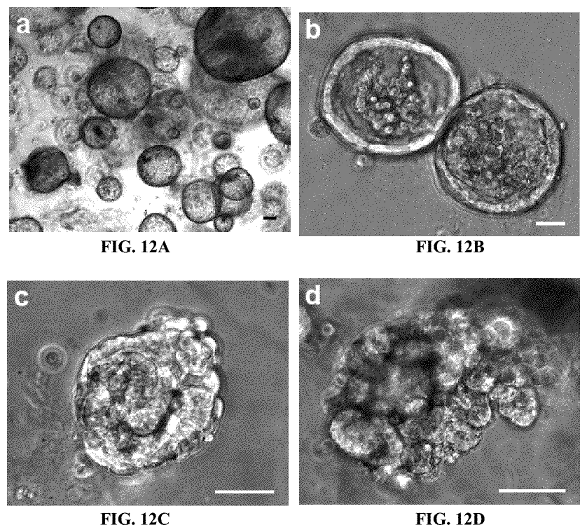

Reference is also made to FIG. 12A-12D, which includes a series of images of expansion and differentiation of equine hepatic organoids. Hepatic organoids were derived from multiple thoroughbred donors and expanded via continual passage.

FIG. 12A includes an image from a representative phase contrast microscopy (low magnification) of UKEOP0004 equine hepatic organoids cultured in expansion media. FIG. 12B includes a representative high magnification phase contrast microscopy image of undifferentiated UKEOP0004 hepatic organoids. These are representative of and related to others that were similarly prepared, such as UKEOP0003.

Equine hepatic organoids can be terminally differentiated through withdrawal of specific growth factors at early or late. FIG. 12C includes a representative image of UKEOP0004 hepatic organoids, in which there was an early (passage 4) withdrawal of specific growth factures. FIG. 12D includes a representative image of UKEOP0003 hepatic organoids, in which there was a late (passage 14) withdrawal of specific growth factures.

### Example 6: Characterization of Equine Liver and Kidney Organoids

Immunohistochemistry, histology, and microscopy techniques will be used to assess the kidney tubuloid differentiation, morphology, and architecture. In addition, gene expression patterns will be analyzed by qPCR probing for markers of proximal tubule formation including apically-localized acetylated tubulin, production of Na+/K+-ATPase (AT1A1), Ezrin (EZR), CDH1 adherens junctions, and ZO-1 (tight junctions).

The formation of leak-proof tubules will be functionally verified by monitoring diffusion of 20 kDa fluorescein isothiocyanate-dextran throughout the cellular tube compartment by fluorescence microscopy and plate reader-based assays. Additional functional studies that will be focused on trans-epithelial drug transport will be adapted to 3D organoid culture.

In addition to qPCR and histological analysis of hepatic and cholangiocyte organoids, several biochemical techniques will be used to validate hallmarks of hepatic function including the production of albumin, urea secretion, bile acid conversion, and cytochrome P450 functionality. Analytical chemistry and liquid chromatography-mass spectrometry will be used to analyze and quantify the production of known drug metabolites produced by 3D equine organoids in response to physiologic drug concentrations.

Collectively, these studies will establish the use and application of equine hepatic organoids for pharmacology and drug discovery endeavors. In addition, equine hepatic organoids will be used to investigate regenerative tissue morphogenesis, infection dynamics of hepatotropic viruses, and immune-modulated disease. Equine liver organoids will be used to prepare an equine liver-on-a-chip system.

### Example 7: Equine Stem Cell Culture and Analysis

With consideration to protocols for human and mouse organoid systems and protocols used to generate stem cell-derived organoids from diverse mammalian origins, methods to generate equine liver, kidney, respiratory, and gastrointestinal (GI) tissue were developed and validated. Completed work includes generating organoids from two thoroughbred foals and two mixed breed juvenile horses. In order to increase the genetic diversity of our organoid platform, organoids will be derived from additional breeds and thoroughbred donors of various ages.

For these studies, healthy post-mortem tissue will be obtained from horses humanely euthanized for reasons unrelated to the study. Donor tissue will be immediately processed and dissociated for progenitor cell enrichment. Isolated stem cells and basal cells will be seeded in a culture apparatus containing basement membrane matrix and growth factors specific to the tissue microenvironment of the native organ. When necessary, equine endothelial support cells will be co-cultured with tissue-specific basal and stem cells. Remaining tissue will be processed for cryobanking in order to maintain a supply of healthy tissue for future investigations.

Equine organoids differentiation into expected cell types will be verified via paraffin-embedded tissue sectioning, immunohistochemistry, and microscopy to analyze specific cell surface markers, as well as qPCR analysis targeting transcripts associated with specific differentiated epithelial cells. Organoid differentiation and tissue morphology will be verified by a board-certified pathologist.

In addition to developing lineages of organoids from healthy donor tissue, in order to develop specific disease models, organoids will be generated from horses diagnosed with conditions that have documented genetic components, such as recurrent airway obstruction.

The physiology of diseased and healthy organoids will be compared using protocols established for human disease organoid models. In parallel studies, the possibility will be explored of generating equine organoids from non-invasive sampling procedures such as rectal swabs, nasal brushings, and urine collection.

Previous work in human patients has demonstrated the ability to generate kidney and bladder organoids from stem cells shed in the urine. Likewise, nasal brush biopsies and rectal swabs have been shown to retrieve sufficient progenitor cell populations for organoid derivation. Non-invasive brush biopsies and rectal swabs will be obtained from healthy donors of various ages and breeds, and culturing protocols referenced herein will be used to derive 3D organoids. Biopsy- and urine-derived organoids will be used to perform a variety of comparative studies evaluating differences in drug metabolism, susceptibility to infectious disease, and tissue longevity.

In addition to expanding the genetically diverse organoid library, these studies are relevant to equine personalized medicine.

### Example 8: Analysis of Infectious Disease Dynamics in Lung Organoid Models

In order to interrogate mechanisms underlying viral and bacterial colonization of the equine lung prior to in vivo challenge studies, an in vitro infection model will be developed using differentiated lung organoids. Protocols were established to enrich bronchioalveolar basal cells and transition these cells from 3D to 2D for expansion.

In these studies, transwell culture systems will be seeded with airway organoid-derived basal cells. Transwell cultures will be transitioned to an air-liquid interface to drive epithelial differentiation and polarization. In this format, cellular receptors localized to the apical surface of the airway epithelium will be accessible to viral and bacterial pathogens inoculated into the well. This infection challenge model will also allow for more precise control of the multiplicity of infection (MOI), and will permit the collection of cell culture media from both the apical and basolateral chambers for tittering and cytokine analysis.

Using the transwell system, the ability of organoid-derived cells to support EIV and EHV-1 infection and produce the appropriate cytokine response will be evaluated. Polarized equine respiratory epithelial cells will be challenged by either WT or mutant strains, and infection kinetics, antiviral responses, and cytokine production will be analyzed by ELISA and qPCR. Subsequent studies will employ live cell microscopy and dual host-pathogen genome-wide transcriptomic studies to provide insight into viral pathogenesis, dissect complex cross-talk between the host and pathogen, and explore mechanisms governing viral infection.

In parallel, bronchioalveolar organoids and differentiated polarized respiratory epithelial cell cultures will be used to develop an in vitro model of R. equi infection. The experimental design will mirror the natural route of infection marked by infiltration of R. equi-parasitized alveolar macrophages into bronchioalveolar tissue.

In the lung, R. equi creates a protected replicative niche within alveolar macrophages where the bacterium remains shielded from antibiotic intervention and subverts antibody-mediated elimination. It is contemplated that, in addition to elements encoded on virulence-associated plasmids, R. equi harbors chromosomal genes that are required for tissue tropism, lung colonization, and pulmonary lesion formation. Using in vivo transposon mutagenesis strategies, an R. equi genomic library was generated in a well-characterized strain (R. equi 103+) that can be used in subsequent foal challenge studies.

In initial in vitro infection studies, this genomic library will be used to investigate the ability of R. equi mutants to colonize lung organoids and to identify bacterial pathogenicity determinants that can be therapeutically targeted in new treatment and prevention strategies. Primary equine alveolar macrophages obtained via bronchoalveolar lavage will be infected in vitro using a pool of individual R. equi transposon insertion mutants. R. equi-containing macrophages will be inoculated into the basolateral chamber of 3D air-liquid interface cultures to permit chemotactic migration and infiltration into proximal lung organoids.

Macrophage infiltration will be monitored via live cell microscopy. Bacterial colonization dynamics will be assessed using established protocols to quantify bacterial viability and to analyze pro-inflammatory cytokine production. Immunoblotting and kinetic assays will be used to investigate the mechanisms underlying R. equi-induced tissue injury and death.

In addition, lung organoid infection models will be used to conduct transcriptome-wide studies that (i) resolve host pathways that govern lung infection biology and (ii) delineate bacterial genetic programs that are required for pathogen survival within the intracellular niche. Respiratory organoid infection models will be used to analyze R. equi biogeography within lung tissue and to evaluate bacterial susceptibility to promising antimicrobials.

### Example 9: Equine Proximal Airway Models of Viral Infection

Equine bronchioalveolar organoids were dissociated into single cell suspensions that were subsequently seeded onto a 0.4 μm pore transwell apparatus. Organoid-derived basal and stem cells were cultured under air-liquid interface for 28 days to allow cellular differentiation. Monolayers were infected with either equine influenza virus A (EIV [A/equine/Ohio/03, Florida sublineage clade 1]) at a multiplicity of infection (MOI) of 0.1 or 1 virus per equine cell, or equine herpes virus 1 (EHV-1 [neuropathogenic T953 strain]) at an MOI of 5 viruses per cell. As a control, transwells were mock infected and cultured for the duration of the experiment.

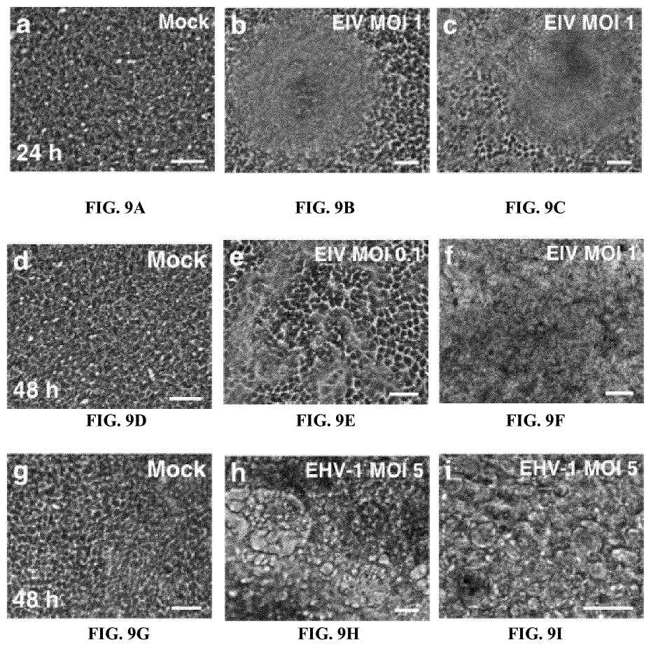

At 24 h and 48 h, infected and mock-infected transwells were imaged by phase contrast microscopy to evaluate viral-induced cytopathic effects. Compared to mock-infected airway models (FIG. 9A), cytopathic effects and clear viral plaques were observed in EIV-infected transwells at an MOI of 1 (FIG. 9B and FIG. 9C) by 24 h post-infection.

At 48 h, mock-infected equine transwells (FIG. 9D) displayed tight junctions and normal morphology compared to significant disruption of cellular junctions and plaque formation observed in transwells infected at an MOI of 0.1 (FIG. 9E), and complete tissue destruction at an MOI of 1 (FIG. 9F). In contrast to standard two-dimensional Madin-Darby Canine Kidney (MDCK) cell culture-based approaches used to study EIV pathogenesis, organoid-derived transwells did not require the addition of tosylsulfonyl phenylalanyl chloromethyl ketone (TPCK)-trypsin to facilitate EIV entry and replication.

Compared to mock-infected transwells (FIG. 9G), proximal airway organoid-derived models supported EHV-1 replication, with intracellular cell-to-cell spreading observed at an MOI of 5 (FIG. 9H and FIG. 9I) by 48 h post-viral challenge. The observed cytopathic effects of EIV and EHV-1 infection were markedly different (plaques vs. intracellular viral dissemination). These studies demonstrate the capacity of equine bronchioalveolar organoid-derived airway models to support kinetics of both rapid (EIV) and slow (EHV-1) viral replication.

FIG. 9A-9I include images from UKEOP0001, but are representative of and related to others that were similarly prepared, such as UKEOP0002.

### Example 10: Identification and Quantification of Ciclesonide Metabolites Produced by Equine Airway and Hepatic Organoid-Derived Tissue Systems

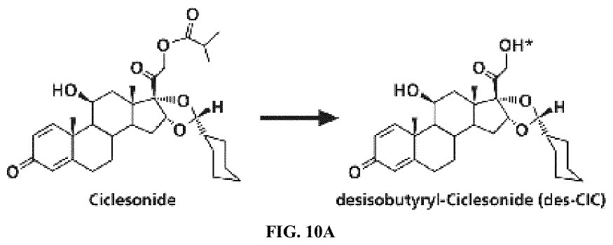

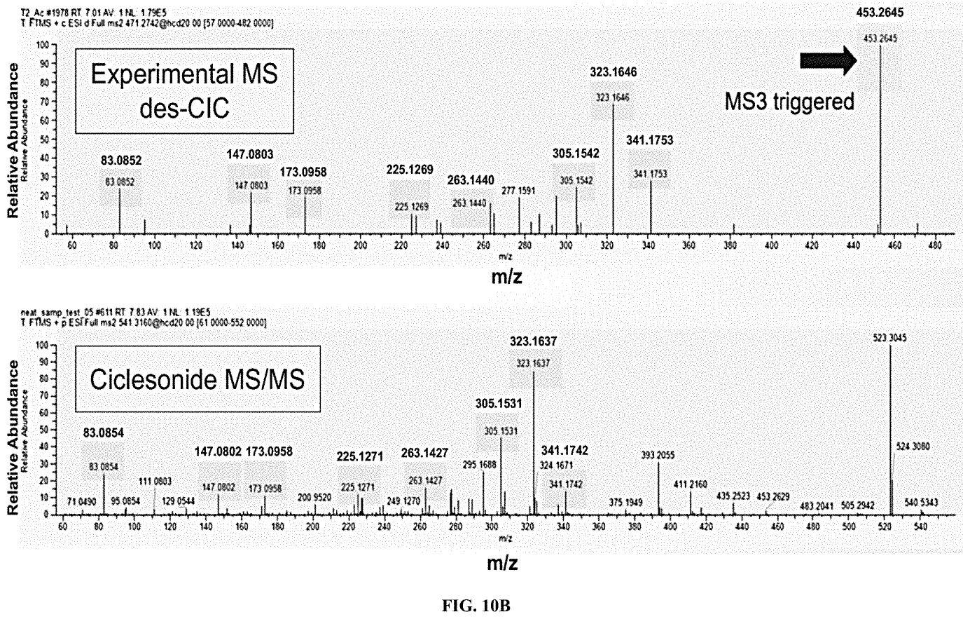

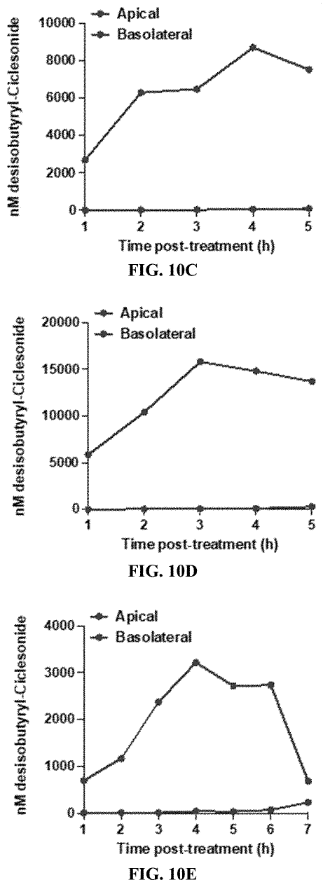

Ciclesonide is an inhaled glucocorticosteroid prescribed in equids for treatment of severe asthma and related airway complications. With reference to FIG. 10A, the Ciclesonide pro-drug is metabolized in the lungs (target organ) to the active form, desisobutyryl-Ciclesonide (des-CIC). The parental pro-drug and the active metabolite exhibit similar fragmentation patterns leading to difficulties in compound differentiation by mass spectrometry (MS) approaches.

Compound optimizations were conducted for Ciclesonide and des-CIC, as well as the internal standard desisobutyryl-ciclesonide-d¹¹, using the Thermo Orbitrap Exploris™ 480™ and Orbitrap IQ-X™ Tribrid™ platforms. Targeted MS methods for each instrument were employed. The Exploris™ 480 employed tMS2 acquisitions, whereas the IQ-X™ Tribrid™ employed Met-IQ Real-Time Library Searching (RTLS) and AcquireX for targeted mass exclusion. Both methods use library comparison technologies, wherein the experimental fragmentation data is compared to the previously acquired library data for compound identifications. Background exclusion transitions were performed using samples generated from untreated equine airway and hepatic organoid-derived transwell systems.

FIG. 10B, includes representative fragmentation pattern of kinetic des-CIC conversion from CIC hydrolysis generated by equine bronchioalveolar organoid-derived transwells treated with pro-drug. The observed MS³ spectra provided additional confirmation of the primary metabolite des-CIC. Quantification of des-CIC metabolite produced by equine airway organoid-derived transwells treated with 50 μM (FIG. 10C) or 500 μM (FIG. 10D) Ciclesonide over time. FIG. 10B-10D include data obtained from UKEOP0001, but are representative of and related to others that were similarly prepared, such as UKEOP0002.

Apical samples were obtained from the transwell upper chamber containing the bronchioalveolar tissue; basolateral samples were obtained from the lower transwell chamber containing untreated medium. FIG. 10E includes the results of quantification of des-CIC metabolite produced by equine hepatic organoid-derived transwells treated with 500 μM Ciclesonide. FIG. 10E include data obtained from UKEOP0003, but are representative of and related to others that were similarly prepared, such as UKEOP0004.

Sample collection was performed from both apical and basolateral transwell chambers as described for airway organoid-derived transwell systems. This study confirms that equine hepatic and airway organoid-derived tissue models produce cellular esterase(s) that have the capacity to hydrolyze Ciclesonide pro-drug to the active desisobutyryl-Ciclesonide metabolite. Further, pro-drug hydrolysis by both equine organoid-derived tissue culture models was rapid and linear.

### Example 11: Horse-On-a-Chip

Mechanical forces, such as fluidic shear stress and lateral strain, are critical for driving in vivo-relevant biology. Biomechanical forces can influence gene expression, cellular function, cell shape, and tissue architecture. To generate a physiologically-relevant system in which to study equine tissues in the context of biomechanics, the equine organoid models will be translated into an innovative microfluidic chip-based platform that recapitulates the native tissue microenvironment in the horse. Indeed, as disclosed herein, multiple, distinct organoid-derived cells have been translated into microfluidic organ-chips that recapitulate tissue microarchitecture and incorporate the mechanical stress, e.g., of a breathing lung and intestinal peristalsis. Examples of equine organoids produced as described herein include the following.

| Organoid Type | ID | Donor |
|---|---|---|
| Bronchioalveolar Organoids | UKEOP0001 | TB 1 |
| Tracheospheres | UKEOP0002 | TB 1 |
| Hepatic Organoids | UKEOP0003 | TB 2 |
| Hepatic Organoids | UKEOP0004 | TB 3 |
| Kidney Tubuloids | UKEOP0005 | TB 2 |
| Gastric Organoids | UKEOP0006 | TB 2 |
| Duodenal Enteroids | UKEOP0007 | TB 2 |
| Jejunal Enteroids | UKEOP0008 | LL01 |
| Jejunal Enteroids | UKEOP0009 | LL02 |
| Jejunal Enteroids | UKEOP0010 | LL03 |
| Jejunal Enteroids | UKEOP0011 | LL04 |
| Jejunal Enteroids | UKEOP0012 | T015 |
| Ileal Enteroids | UKEOP0013 | TB 2 |

Equine organoid systems will be translated into a new horse organ-on-a-chip platform that more precisely reflects native organ function.

Engineered microfluidic chips are designed to facilitate biologically-relevant interactions between extracellular matrices and host-derived cells; maintain native cell architecture; enable tissue-tissue interactions; introduce stretch and fluid flow to recreate biomechanical forces; and offer the ability to introduce resident and circulating immune cells (such as bronchoalveolar macrophages). For example, Emulate, Inc. manufactures advanced cell culture devices that enable the user to recreate biomechanical forces with virtually any cell source while maintaining relevant tissue-tissue interactions (FIG. 3 and FIG. 5). The Emulate system incorporates unilateral flow and mechanical strain within stretchable, optically clear microfluidic chips that can be directly imaged on multiple microscopy platforms. The platform offers the ability to interface multiple tissue types to reproduce the native organ environment. Of note, the response from tissues in an organ-chip environment have been shown to more faithfully recapitulate an in vivo response and thus serve as a valuable surrogate for translational studies.

As disclosed herein, a lung-on-a-chip (FIG. 3) and a small intestine-on-a-chip (FIG. 5) were developed, derived from equine organoids. The flexible nature of the chip allows for the application of rate-controlled mechanical stretching forces to simulate breathing and peristalsis. The equine lung-on-a-chips will be validated and used to monitor respiratory infection kinetics of EIV, EHV, and R. equi via live-cell confocal microscopy.

One major advantage of organ-chip technology is the design of the chip membrane that includes a pattern of 7 μm pores to allows for transmigration of immune cells from the vasculature channel into the epithelial channel. This system will therefore allow for engineering of complex in vitro models of respiratory infectious disease pathogenesis that incorporate multiple tissue types, defined immune cell infiltration, fluid flow through microvasculature, an air-liquid interface, and relevant biomechanical forces.

With microfluidic chip technology, endpoint experiments that can be measured in other cell culture-based systems (such as RNAseq, cytokine analysis, histology, microscopy, etc.) can be readily adapted and analyzed. Therefore, infection kinetics of lung-chip tissues will be analyzed by monitoring a variety of parameters including cytokine production, viral replication kinetics, levels of bacterial proliferation, and global transcriptomic/proteomic analyses. In addition to comparing lung-chip cellular responses to WT and mutant pathogen challenge, the impact of mechanical stretch and fluid flow to infection efficiency and cytokine production will be analyzed.

Likewise, the intestine-on-a-chip will be used to build a biologically-relevant in vitro model of Salmonella-induced enteritis. Because the intestine-on-a-chip is a ‘germ-free’ system, the model can be used to isolate Salmonella-specific tissue responses. The ability of intestine-chips to support colonization by equine-specific microbiota will also be evaluated, to perform studies aimed at understanding how invasive pathogens disrupt the resident flora to trigger colic. These studies may also identify a protective microbiome that guards against severe gastrointestinal disease.

The chip-based investigations will rely on the application of directional, rate-controlled fluid flow coupled to stretching patterns that mimic peristalsis. Collectively, these studies will delineate the contribution of biomechanical forces in equine infection pathobiology.

Hepatic and biliary organoids will be integrated into a novel equine liver-on-a-chip. Optimized protocols have been developed for the seamless transition of human organoid culture to organ-on-a-chip platforms, and these protocols have been successfully modified for use in equine organoid culture. Specifically, liver chips will integrate organoid-derived hepatocytes interfaced with endothelial cells coupled to relevant cytoarchitecture and physiological flow. Differentiation of hepatocytes will be validated using qPCR and immunohistochemistry techniques.

Taking advantage of the unilateral flow within organ-chips, equine liver-chips will be used for drug metabolism studies. The application of equine liver-chips will be validated for toxicology and drug metabolism studies using therapeutics with known pharmacokinetics and metabolite profiles. For these experiments, therapeutics of interest will be added to the inlet chamber, pumped through liver-tissue channels at a physiologically-defined flow rate that facilitates drug metabolism, and collected in dedicated effluent chambers. Analytic chemistry and metabolite identification will be performed via mass spectrometry.

The innovative design of the organ-chip and culture housing permits scheduled sampling of the independent inlet and effluent chambers, and uninterrupted directional flow through the microfluidic chips ensures that all cells are exposed to physiologic concentrations of the assayed drug. Importantly, the open system permits continuous collection or sampling of the effluent of both vascular and parenchymal channels, which facilitates recovery of both parent and metabolite(s) biological endpoints over time. Thus, the equine hepatic organoid and liver-on-a-chip platform represents a unique and high-impact pipeline that will enable translational biomedical research focused on understanding drug metabolism in the horse.

Together, the ‘horse-on-a-chip’ will provide a dynamic, physiologically-relevant platform for the study of equine-specific disease mechanisms and metabolic flux in the context of diverse, multi-tissue microenvironments and native tissue architectures.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Baumler, A. & Fang, F. C. Host specificity of bacterial pathogens. Cold Spring Harb Perspect Med 3, a010041, doi:10.1101/cshperspect.a010041 (2013).
Olival, K. J. et al. Host and viral traits predict zoonotic spillover from mammals. Nature 546, 646-650, doi:10.1038/nature22975 (2017).
Woolhouse, M. E. & Gowtage-Sequeria, S. Host range and emerging and reemerging pathogens. Emerg Infect Dis 11, 1842-1847, doi:10.3201/eid1112.050997 (2005).
Jones, K. E. et al. Global trends in emerging infectious diseases. Nature 451, 990-993, doi:10.1038/nature06536 (2008).
Plowright, R. K. et al. Pathways to zoonotic spillover. Nat Rev Microbiol, doi:10.1038/nrmicro.2017.45 (2017).
Morse, S. S. et al. Prediction and prevention of the next pandemic zoonosis. Lancet 380, 1956-1965, doi:10.1016/S0140-6736(12)61684-5 (2012).
Stewart, A. S., Freund, J. M. & Gonzalez, L. M. Advanced three-dimensional culture of equine intestinal epithelial stem cells. Equine Vet J 50, 241-248, doi:10.1111/evj.12734 (2018).
Chandra, L. et al. Derivation of adult canine intestinal organoids for translational research in gastroenterology. BMC Biol 17, 33, doi:10.1186/s12915-019-0652-6 (2019).
Derricott, H. et al. Developing a 3D intestinal epithelium model for livestock species. Cell Tissue Res 375, 409-424, doi:10.1007/s00441-018-2924-9 (2019).
Lipsitch, M. et al. Viral factors in influenza pandemic risk assessment. Elife 5, doi:10.7554/eLife.18491 (2016).
Zhou, J. et al. Differentiated human airway organoids to assess infectivity of emerging influenza virus. Proc Nat Acad Sci USA 115, 6822-6827, doi:10.1073/pnas.1806308115 (2018).
Kim, C. F. et al. Identification of bronchioalveolar stem cells in normal lung and lung cancer. Cell 121, 823-835, doi:10.1016/j.cell.2005.03.032 (2005).
Rock, J. R. et al. Basal cells as stem cells of the mouse trachea and human airway epithelium. Proc Nat Acad Sci USA 106, 12771-12775, doi:10.1073/pnas.0906850106 (2009).
Lee, J. H. et al. Lung stem cell differentiation in mice directed by endothelial cells via a BMP4-NFATc1-thrombospondin-1 axis. Cell 156, 440-455, doi:10.1016/j.cell.2013.12.039 (2014).
Zhang, H. et al. Lkb1 inactivation drives lung cancer lineage switching governed by Polycomb Repressive Complex 2. Nat Commun 8, 14922, doi:10.1038/ncomms14922 (2017).
Barkauskas, C. E. et al. Lung organoids: current uses and future promise. Development 144, 986-997, doi:10.1242/dev.140103 (2017).
Jain, A. et al. Primary human lung alveolus-on-a-chip model of intravascular thrombosis for assessment of therapeutics. Clin Pharmacol Ther 103, 332-340, doi:10.1002/cpt.742 (2018).
Benam, K. H. et al. Matched-comparative modeling of normal and diseased human airway responses using a microengineered breathing lung chip. Cell Syst 3, 456-466 e454, doi:10.1016/j.cels.2016.10.003 (2016).
Huh, D. et al. A human disease model of drug toxicity-induced pulmonary edema in a lung-on-a-chip microdevice. Sci Transl Med 4, 159ra147, doi:10.1126/scitranslmed.3004249 (2012).
Huh, D. et al. Microfabrication of human organs-on-chips. Nat Protoc 8, 2135-2157, doi:10.1038/nprot.2013.137 (2013).
Peel, S. et al. Introducing an automated high content confocal imaging approach for organs-on-chips. Lab Chip 19, 410-421, doi:10.1039/c81c00829a (2019).
22. Parker, et al., U.S. Pat. No. 9,857,356 for “Muscle chips and methods of use thereof.”
23. Wikswo, et al., U.S. Pat. No. 9,725,687 for “Integrated human organ-on-chip microphysiological systems.”
24. Wikswo, et al., U.S. Pat. No. 10,078,075 for “Integrated organ-on-chip systems and applications of the same.”
25. Ingber, et al., U.S. Pat. No. 10,087,422 for “Organ chips and uses thereof.”
26. Kerns, et al., U.S. Patent Application Publication No. 2019/0031992 for “Systems and methods for growth of intestinal cells in microfluidic devices.”
27. Date and Sato, Mini-Gut Organoids: Reconstitution of the Stem Cell Niche. Annual Review of Cell and Developmental Biology 31:1, 269-289 (2015).
28. Kasendra, et al., Duodenum Intestine-Chip for preclinical drug assessment in a human relevant model. Cell Biology, eLife 2020; 9:e50135 doi: 10.7554/eLife.50135 (2020).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of culturing cells, comprising:
a) obtaining equine primary cells derived from equine tissue selected from the group consisting of lung, trachea, stomach, small intestine duodenal, small intestine ileal, liver, bile duct, kidney, bone, skin, pancreas, cecum, colon, brain, neuron, salivary gland, retina, placenta, uterus, and mammary gland; and
b) culturing the equine primary cells to promote self-assembly, formation, and differentiation of one or more organoids.

2. The method of claim 1, and further comprising obtaining equine primary cells derived from equine small intestine jejunal tissue.

3. The method of claim 1, wherein the equine tissue includes tissue selected form the group consisting of: lung, trachea, glandular stomach, non-glandular stomach, bile duct cholangiocyte, small intestine duodenal, small intestine jejunal tissue, small intestine ileal, liver hepatocyte, kidney epithelial tubuloid, bone, skin, brain, salivary gland, retina, placenta, uterus, mammary gland.

4. A method of preparing an in vitro equine organ model system, comprising:
a) providing a microfluidic device comprising an upper chamber and a lower chamber, separated by a membrane that permits the exchange of cellular signals and soluble molecules;
b) dissociating a three-dimensional organoid comprising multiple cell types, wherein the organoid was prepared using equine tissue associated with an organ of interest;
c) selecting equine primary cells from the dissociated organoid; [without using an antibody for cell sorting, as was necessary in prior art human organ on a chip devices]
d) seeding the upper chamber of the device with the equine primary cells;
e) expanding the equine primary cells in a submerged two-dimensional adherent cell culture; and
f) differentiating the equine primary cells to create differentiated cell types associated with the organ of interest.

5. The method of claim 4, and further comprising seeding the lower chamber with equine endothelial cells.

6. The method of claim 4, wherein expanding the equine primary cells further comprises applying fluid flow, thereby initiating differentiation of the equine primary cells.

7. The method of claim 6, wherein the organ(s) of interest is from: (a) the airway, and further comprising applying air flow and mechanical movement to obtain to obtain a pseudostratified epithelium; (b) the intestine, and further comprising continued application of fluid flow and applying mechanical movement to obtain a pseudostratified epithelium; (c) the liver, and further comprising continued application of fluid flow to obtain a pseudostratified epithelium; or (d) the kidney, and further comprising continued application of fluid flow to obtain a pseudostratified epithelium.

8. The method of claim 4, and further comprising seeding the upper chamber with stem cells of the organ(s) of interest.

9. The method of claim 4, wherein the equine primary cells are from a tissue or organoid selected from the group consisting of lung, trachea, stomach, intestine, liver, bile duct, kidney, bone, skin, pancreas, cecum, colon, brain, neuron, salivary gland, retina, placenta, uterus, and mammary gland.

10. The method of claim 4, wherein the organ of interest comprises one or more respiratory system organs, one or more gastrointestinal tract organs, one or more hepatic system organs, or one or more renal (urinary) system organs.

11. The method of claim 4, wherein the equine primary cells are stem cells.

12. The method of claim 11, wherein the stem cells are airway stem cells, intestinal stem cells, hepatic stem cells, or renal stem cells.

13. A kit for an in vitro equine organ model system, comprising an organoid prepared using equine tissue associated with the organ of interest; or equine primary cells, wherein the equine primary cells are derived from equine tissue associated with an organ of interest, or derived from an organoid prepared using equine tissue associated with the organ of interest.

14. The kit of claim 13, and further comprising regents culturing the equine primary cells.

15. The kit of claim 14, wherein the reagents comprise components for expanding and/or differentiating the cells.

16. The kit of claim 15, wherein the reagents comprise cell culture media.

17. The kit of claim 16, wherein the reagents further comprise growth factors specific to a tissue microenvironment of the organ of interest.

18. The kit of claim 13, wherein the organoid is selected from the group consisting of UKEOP0001, UKEOP0002, UKEOP0003, UKEOP0004, UKEOP0005, UKEOP0006, UKEOP0007, UKEOP0008, UKEOP0009, UKEOP0010, UKEOP0011, UKEOP0012, and UKEOP0013.

19. The kit of claim 13, and further comprising a microfluidic device comprising an upper chamber and a lower chamber, separated by a membrane that permits the exchange of cellular signals and soluble molecules

20. The kit of claim 13, and further comprising a culture apparatus containing basement membrane matrix
